(19) United States
(12) Patent Application Publication
(10) Pub. No.: US 2003/0022184 A1
(43) Pub. Date: Jan. 30, 2003

(54) Coding sequences of the human BRCA1 gene

(75) Inventors: Patricia D. Murphy, Slingerlands, NY (US); Antonette C.P. Allen, Severn, MD (US); Christopher P. Alvares, Potomac, MD (US); Brenda S. Critz, Frederick, MD (US); Sheri J. Olson, Falls Church, VA (US); Denise Thurber, Silver Spring, MD (US); Bin Zeng, Indianapolis, IN (US)

Correspondence Address: MORGAN LEWIS & BOCKIUS LLP, 1111 PENNSYLVANIA AVENUE NW, WASHINGTON, DC, 20004, US

(73) Assignee: OncorMed. Inc.

(21) Appl. No.: 09/982,828

(22) Filed: Oct. 22, 2001

Related U.S. Application Data
(62) Division of application No. 09/074,453, filed on May 6, 1998.
Application No. 09/074,453 is a continuation-in-part of application No. 08/798,691, filed on Feb. 12, 1997.
Application No. 08/798,691 is a continuation-in-part of application No. 08/598,591, filed on Feb. 12, 1996.

(51) Int. Cl. C12Q 1/68; C07H 21/04; C12N 9/00; C12M 1/34

(52) U.S. Cl. 435/6; 435/287.2; 435/183; 536/24.3

(57) ABSTRACT

This invention is directed to the isolated coding sequences and to the protein sequences they code for. This invention is directed to three coding sequence of the BRCA1 gene. The three coding sequences, BRCA1^{(omi1)}, BRCA1^{(omi2)}, and BRCA1^{(omi3)} and their frequencies of occurrence are provided together with the protein sequences they code for. Another aspect of this invention is a method of determining the consensus sequence for any gene. Another aspect of the invention is a method of identifying an individual having an increased genetic susceptibility to breast or ovarian cancer because they have inherited a causative mutation in their BRCA1 gene. This invention is also related to a method of performing gene therapy with any of the isolated BRCA1 coding sequences. This invention is further related to protein therapy with BRCA1^{(omi)} proteins or their functional equivalent.

This application is a Continuation-In Part of U.S. application Ser. No. 08/798,691 filed on Feb. 12, 1997, which in turn is a Continuation-In Part of U.S. application Ser. No. 08/598,591 filed on Feb. 12, 1996 and issued on Aug. 5, 1997 as U.S. Pat. No. 5,654,155.

### CODING SEQUENCES OF THE HUMAN BRCA1 GENE

1. Field of the Invention

This invention relates to a gene which has been associated with breast and ovarian cancer where the gene is found to be mutated. More specifically, this invention relates to the three coding sequences of the BRCA1 gene BRCA1^{(omi1)}, BRCA1^{(omi2)}, and BRCA1^{(omi3)} isolated from human subjects.

2. Background of the Invention

It has been estimated that about 5-10% of breast cancer is inherited Rowell, S., et al., American Journal of Human Genetics 55:861-865 (1994). Located on chromosome 17, BRCA1 is the first gene identified to be conferring increased risk for breast and ovarian cancer. Miki et al., Science 266:66-71 (1994). Mutations in this “tumor suppressor” gene are thought to account for roughly 45% of inherited breast cancer and 80-90% of families with increased risk of early onset breast and ovarian cancer. Easton et al., American Journal of Human Genetics 52:678-701 (1993).

Locating one or more mutations in the BRCA1 region of chromosome 17 provides a promising approach to reducing the high incidence and mortality associated with breast and ovarian cancer through the early detection of women at high risk. These women, once identified, can be targeted for more aggressive prevention programs. Screening is carried out by a variety of methods which include karyotyping, probe binding and DNA sequencing.

In DNA sequencing technology, genomic DNA is extracted from whole blood and the coding sequences of the BRCA1 gene are amplified. The coding sequences might be sequenced completely and the results are compared to the DNA sequence of the gene. Alternatively, the coding sequence of the sample gene may be compared to a panel of known mutations before completely sequencing the gene and comparing it to a normal sequence of the gene.

If a mutation in the BRCA1 coding sequence is found, it may be possible to provide the individual with increased expression of the gene through gene transfer therapy. It has been demonstrated that the gene transfer of the BRCA1 coding sequence into cancer cells inhibits their growth and reduces tumorigenesis of human cancer cells in nude mice. Jeffrey Holt and his colleagues conclude that the product of BRCA1 expression is a secreted tumor growth inhibitor, making BRCA1 an ideal gene for gene therapy studies. Transduction of only a moderate percentage of tumor cells apparently produces enough growth inhibitor to inhibit all tumor cells. Arteaga, C L and J T Holt Cancer Research 56: 1098-1103 (1996), Holt, J T et al., Nature Genetics 12: 298-302 (1996). The observation of the BRCA1 growth inhibitor being a secreted protein also leads to the possible use of injection of the BRCA1 growth inhibitor into the area of the tumor for tumor suppression.

The BRCA1 gene is divided into 24 separate exons. Exons 1 and 4 are noncoding, in that they are not part of the final functional BRCA1 protein product. The BRCA1 coding sequence spans roughly 5600 base pairs (bp). Each exon consists of 200-400 bp, except for exon 11 which contains about 3600 bp. To sequence the coding sequence of the BRCA1 gene, each exon is amplified separately and the resulting PCR products are sequenced in the forward and reverse directions. Because exon 11 is so large, we have divided it into twelve overlapping PCR fragments of roughly 350 bp each (segments “A” through “L” of BRCA1 exon 11).

Many mutations and polymorphisms have already been reported in the BRCA1 gene. A world wide web site has been built to facilitate the detection and characterization of alterations in breast cancer susceptibility genes. Such mutations in BRCA1 can be accessed through the Breast Cancer Information Core at:http://www.nchgr.nih.gov/dir/lab_transfer/bic.

This data site became publicly available on Nov. 1, 1995. Friend, S. et al. Nature Genetics 11:238, (1995).

The genetics of Breast/Ovarian Cancer Syndrome is autosomal dominant with reduced penetrance. In simple terms, this means that the syndrome runs through families: (1) both sexes can be carriers (mostly women get the disease but men can both pass it on and occasionally get breast cancer); (2) most generations will likely have breast cancer; (3) occasionally women carriers either die young before they have the time to manifest disease (and yet have offspring who get it) or they never develop breast or ovarian cancer and die of old age (the latter people are said to have “reduced penetrance” because they never develop cancer). Pedigree analysis and genetic counseling is absolutely essential to the proper workup of a family prior to any lab work.

Until now, only a single coding sequence for the BRCA1 gene has been available for comparison to patient samples. That sequence is available as GenBank Accession Number U14680. There is a need in the art, therefore, to have available a “consensus coding sequence” found in the majority of the normal population and other coding sequences found in normal population. The availability of these coding sequences will make it possible for true mutations to be easily identified or differentiated from polymorphisms. Identification of mutations of the BRCA1 gene and protein would allow more widespread diagnostic screening for hereditary breast and ovarian cancer than is currently possible. In addition, these coding sequences also have utility in gene therapy, protein replacement therapy, and diagnosis.

Knowing the coding sequences which do not represent a higher susceptibility to an individual for cancer will reduce the likelihood of misinterpreting a “sequence variation” found in the population (i.e. polymorphism) with a pathologic “mutation” (i.e. causes disease in the individual or puts the individual at a high risk of developing the disease). With large interest in breast cancer predisposition testing, misinterpretation is particularly worrisome. People who already have breast cancer are asking the clinical question: “is my disease caused by a heritable genetic mutation?” The relatives of the those with breast cancer are asking the question: “Am I also a carrier of the mutation my relative has? Thus, is my risk increased, and should I undergo a more aggressive surveillance program.”

### SUMMARY OF THE INVENTION

The present invention is based on the isolation of three coding sequences of the BRCA1 gene found in human individuals.

It is an object of the invention to provide the most commonly occurring coding sequence of the BRCA1 gene.

It is another object of this invention to provide two other coding sequences of BRCA1 gene.

It is another object of the invention to provide three protein sequences coded by three coding sequences of the BRCA1 gene.

It is another object of the invention to provide a list of the codon pairs which occur at each of seven polymorphic points on the BRCA1 gene.

It is another object of the invention to provide the rates of occurrence for the codons.

It is another object of the invention to provide a method wherein BRCA1, or parts thereof, is amplified with one or more oligonucleotide primers.

It is another object of this invention to provide a method of identifying individuals who carry no mutation(s) of the BRCA1 coding sequence and therefore have no increased genetic susceptibility to breast or ovarian cancer based on their BRCA1 genes.

It is another object of this invention to provide a method of identifying a mutation leading to an increased genetic susceptibility to breast or ovarian cancer.

It is another object of the invention to encompass all or fragments of BRCA1^{(omi)} protein, BRCA1^{(omi)} polypeptides, and functional equivalents thereof.

It is another object of the invention to encompass an anti-BRCA1^{(omi)} protein antibody or an antibody using a BRCA1^{(omi)} polypeptide or a functional equivalent thereof as an immunogen.

It is another object of the invention to encompass prokaryotic or eukaryotic host cells comprising an expression vector having a DNA sequence that encodes for all or a fragment of the BRCA1^{(omi)} protein, a BRCA1^{(omi)} polypeptide, or a functional equivalent thereof.

There is a need in the art for a sequence of the BRCA1 gene and for the protein sequence of BRCA1 as well as for an accurate list of codons which occur at polymorphic points on a sequence. A person skilled in the art will find the present invention useful for:

a) identifying individuals having a BRCA1 gene with no coding mutations, who therefore cannot be said to have an increased genetic susceptibility to breast or ovarian cancer from their BRCA1 genes;

b) avoiding misinterpretation of polymorphisms found in the BRCA1 gene;

c) determining the presence of a previously unknown mutation in the BRCA1 gene;

d) identifying a mutation which increases the genetic susceptibility to breast or ovarian cancer;

e) probing a human sample of the BRCA1 gene by allele to determine the presence or absence of either polymorphic alleles or mutations;

f) performing gene therapy with a suitable BRCA1^{(omi)} gene sequence;

g) performing protein replacement therapy with a suitable BRCA1^{(omi)} protein sequence or a functional equivalent thereof; and

(h) performing diagnosis with a reagent derived from the BRCA1^{(omi)} cDNA and protein sequence.

### BRIEF DESCRIPTION OF THE FIGURE

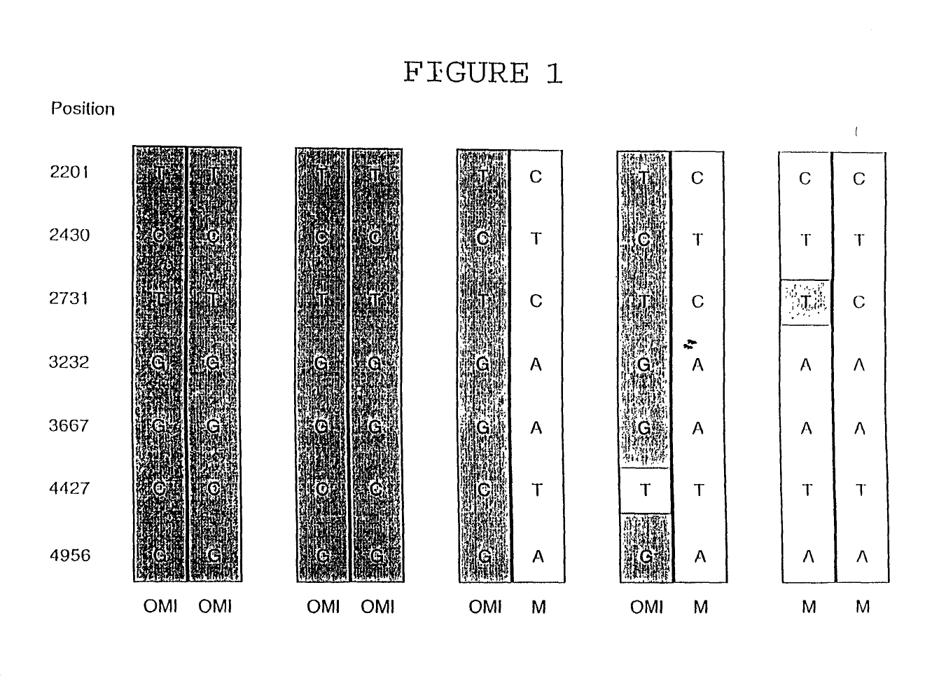

As shown in FIG. 1, the alternative alleles at polymorphic sites along a chromosome which can be represented as a “haplotype” within a gene such as BRCA1. The BRCA1^{(omi1)} haplotype is shown in FIG. 1 with dark shading (encompassing the alternative alleles found at nucleotide sites 2201, 2430, 2731, 3232, 3667, 4427, and 4956). For comparison, the haplotype that is in GenBank is shown with no shading. As can be seen from the figure, the common “consensus” haplotype is found intact in five separate chromosomes labeled with the OMI symbol (numbers 1-5 from left to right). Two additional haplotypes (BRCA1^{(omi2)}, and BRCA1^{(omi3)} are represented with mixed dark and light shading (numbers 7 and 9 from left to right). In total, 7 of 10 haplotypes along the BRCA1 gene are unique.

### DETAILED DESCRIPTION OF THE INVENTION

### Definitions

The following definitions are provided for the purpose of understanding this invention.

“Breast and Ovarian cancer” is understood by those skilled in the art to include breast and ovarian cancer in women and also breast and prostate cancer in men. The BRCA1 gene is also associated with genetic susceptibility to colon cancer. Therefore, claims and specification in this document which recite breast and/or ovarian cancer refer to breast, ovarian, prostate, and colon cancers in men and women.

“Coding sequence” or “DNA coding sequence” refers to those portions of a gene which, taken together, code for a peptide (protein), or which nucleic acid itself has function.

“Protein” or “peptide” refers to an amino acids sequence which has function.

“BRCA1^{(omi)}” refers collectively to the “BRCA1^{(omi1)}”, “BRCA1^{(omi2)}” and “BRCA1^{(omi3)}” coding sequences.

“BRCA1^{(omi1)}” refers to SEQ. ID. NO.:1, a coding sequence for the BRCA1 gene. The coding sequence was found by end to end sequencing of BRCA1 alleles from individuals randomly drawn from a Caucasian population found to have no family history of breast or ovarian cancer. The sequenced gene was found not to contain any mutations. BRCA1^{(omi1)} was determined to be a consensus sequence by calculating the frequency with which the coding sequence occurred among the sample alleles sequenced.

“BRCA1^{(omi2)}” and “BRCA1^{(omi3)}” refer to SEQ. ID. NO.:3 and SEQ. ID. NO.:5 respectively. They are two additional coding sequences for the BRCA1 gene which were also isolated from individuals randomly drawn from a Caucasian population found to have no family history of breast or ovarian cancer.

“Polymorphism” refers to a base change in a DNA sequence which is not associated with known pathology.

“Primer” as used herein refers to a sequence comprising about 15 or more nucleotides having a sequence complementary to the BRCA1 gene. Other primers which can be used for hybridization will be known or readily ascertainable to those skilled in the art.

“Genetic susceptibility” refers to the susceptibility to breast or ovarian cancer due to the presence of a mutation in the BRCA1 gene.

“Target polynucleotide” refers to the nucleic acid sequence of interest e.g., the BRCA1 encoding polynucleotide.

“Consensus” means the most commonly occurring in the population.

“Consensus genomic sequence” means the allele of the target gene which occurs with the greatest frequency in a population of individuals having no family history of disease associated with the target gene.

“Substantially complementary to” refers to probes or primer sequences which hybridize to the sequences provided under stringent conditions and/or sequences having sufficient homology with BRCA1 sequences, such that the allele specific oligonucleotide probe or primers hybridize to the BRCA1 sequences to which they are complimentary.

“Haplotype” refers to a series of alleles within a gene on a chromosome.

“Isolated” refers to substantially free of other nucleic acids, proteins, lipids, carbohydrates or other materials with which they may be associated. Such association is typically either in cellular material or in a synthesis medium.

“Mutation” refers to a base change or a gain or loss of base pair(s) in a DNA sequence, which results in a DNA sequence which codes for a non-functioning protein or a protein with substantially reduced or altered function.

“Biological sample” or “body sample” refers to a sample containing DNA obtained from a biological source. The sample may be from a living, dead or even archeological source from a variety of tissues and cells. Examples include body fluid (e.g. blood (leukocytes), urine (epithelial cells), saliva, breast milk, menstrual flow, cervical and vaginal secretions, etc.), skin, hair roots/follicle, mucus membrane (e.g. buccal or tongue cell scrapings), cervicovaginal cells (from PAP smear, etc.), lymphatic tissue, internal tissue (normal or tumor).

“Vector” refers to any polynucleotide which is capable of self replication or inducing integration into a self-replicating polynucleotide. Examples include polynucleotides containing an origin or replication or an integration site. Vectors may be intergrated into the host cell's chromosome or form an autonomously replicating unit.

“A BRCA1 tumor growth inhibitor” refers to a molecule such as, all or a fragment of BRCA1^{(omi)} protein, a BRCA1^{(omi)} polypeptide, or a functional equivalent thereof that is effective for preventing the formation of, reducing, or eliminating a transformed or malignant phenotype of breast or ovarian cancer cells.

“A BRCA1^{(omi)} polypeptide” refers to a BRCA1 polypeptide either directly derived from the BRCA1^{(omi)} protein, or homologous to the BRCA1^{(omi)} protein, or a fusion protein thereof.

“A functional equivalent” refers to a molecule including an unnatural BRCA1^{(omi)} polypeptide, a drug, or a natural product which retains substantial biological activity as the native BRCA1^{(omi)} protein in preventing, diagnosing, monitoring, and treating breast and ovarian cancer.

The invention in several of its embodiments includes: isolated DNA sequences of the BRCA1^{(omi)} coding sequences as set forth in SEQ ID NO:1, 3 and 5, protein sequence sof the BRCA1^{(omi)} protein sas set forth in SEQ ID NO:2, 4, and 6, a method of identifying individuals having a mutant or normal BRCA1 gene, a method of detecting an increased genetic susceptibility to breast and ovarian cancer in an individual resulting from the presence of a mutation in the BRCA1 coding sequence, a method of performing gene therapy to prevent or treat a tumor, and a method of protein replacement therapy to prevent or treat a tumor.

### Sequence

Any nucleic acid specimen, in purified or non-purified form, can be utilized as the starting nucleic acid or acids, providing it contains, or is suspected of containing, the specific nucleic acid sequence containing a polymorphic locus. Thus, the process may amplify, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous amplification reaction herein, using the same or different primers may be so utilized. See TABLE II. The specific nucleic acid sequence to be amplified, i.e., the polymorphic locus, may be a fraction of a larger molecule or can be present initially as a discrete molecule, so that the specific sequence constitutes the entire nucleic acid. A variety of amplification techniques may be used such as ligating the DNA sample or fragments thereof to a vector capable of replication or incorporation into a replicating system thereby increasing the number of copies of DNA suspected of containing at least a portion of the BRCA1 gene. Amplification techniques also include so called “shot gun cloning.” It is not necessary that the sequence to be amplified be present initially in a pure form; it may be a minor fraction of a complex mixture, such as contained in whole human DNA.

It should be noted that one need not sequence the entire coding region or even an entire DNA fragment in order to determine whether or not a mutation is present. For example, when a mutation is known in one family member, it is sufficient to determine the sequence at only the mutation site when testing other family members.

DNA utilized herein may be extracted from a body sample, such as blood, tissue material and other biological sample by a variety of techniques such as that described by Maniatis, et. al. in Molecular Cloning:A Laboratory Manual, Cold Spring Harbor, N.Y., p 280-281, 1982). If the extracted sample is impure, it may be treated before amplification with an amount of a reagent effective to open the cells, or animal cell membranes of the sample, and to expose and/or separate the strand(s) of the nucleic acid(s). This lysing and nucleic acid denaturing step to expose and separate the strands will allow amplification to occur much more readily.

For amplification by cloning, the isolated DNA may be cleaved into fragments by a restriction endonuclease or by shearing by passing the DNA containing mixture through a 25 gauge needle from a syringe to prepare 1-1.5 kb fragments. The fragments are then ligated to a cleaved vector (virus, plasmid, transposon, cosmid etc.) and then the recombinant vector so formed is then replicated in a manner typical for that vector.

For a PCR amplification, the deoxyribonucleotide triphosphates DATP, dCTP, dGTP, and dTTP are added to the synthesis mixture, either separately or together with the primers, in adequate amounts and the resulting solution is heated to about 90-100° C. from about 1 to minutes, preferably from 1 to 4 minutes. After this heating period, the solution is allowed to cool, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40° C. Most conveniently the reaction occurs at room temperature. When using thermostable DNA polymerase such as Taq, higher temperature may be used.

The primers used to carry out this invention embrace oligonucleotides of sufficient length and appropriate sequence to provide initiation of polymerization. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it may contain fewer nucleotides.

Primers used to carry out this invention are designed to be substantially complementary to each strand of the genomic locus to be amplified. This means that the primers must be sufficiently complementary to hybridize with their respective strands under conditions which allow the agent for polymerization to perform. In other words, the primers should have sufficient complementarity with the 5′ and 3′ sequences flanking the mutation to hybridize therewith and permit amplification of the genomic locus.

Oligonucleotide primers of the invention are employed in the amplification process which is an enzymatic chain reaction that produces exponential quantities of polymorphic locus relative to the number of reaction steps involved. Typically, one primer is complementary to the negative (−) strand of the polymorphic locus and the other is complementary to the positive (+) strand. Annealing the primers to denatured nucleic acid followed by extension with an enzyme, such as the large fragment of DNA polymerase I (Klenow) and nucleotides, results in newly synthesized +and−strands containing the target polymorphic locus sequence. Because these newly synthesized sequences are also templates, repeated cycles of denaturing, primer annealing, and extension results in exponential production of the region (i.e., the target polymorphic locus sequence) defined by the primers. The product of the chain reaction is a discreet nucleic acid duplex with termini corresponding to the ends of the specific primers employed.

The oligonucleotide primers of the invention may be prepared using any suitable method, such as conventional phosphotriester and phosphodiester methods or automated embodiments thereof. In one such automated embodiment, diethylphosphoramidites are used as starting materials and may be synthesized as described by Beaucage, et al., Tetrahedron Letters, 22:1859-1862, 1981. One method for synthesizing oligonucleotides on a modified solid support is described in U.S. Pat. No. 4,458,066.

The agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase, polymerase muteins, reverse transcriptase, other enzymes, including heat-stable enzymes (e.i., those enzymes which perform primer extension after being subjected to temperatures sufficiently elevated to cause denaturation), such as Taq polymerase. Suitable enzyme will facilitate combination of the nucleotides in the proper manner to form the primer extension products which are complementary to each polymorphic locus nucleic acid strand. Generally, the synthesis will be initiated at the 3′ end of each primer and proceed in the 5′ direction along the template strand, until synthesis terminates, producing molecules of different lengths.

The newly synthesized strand and its complementary nucleic acid strand will form a double-stranded molecule under hybridizing conditions described above and this hybrid is used in subsequent steps of the process. In the next step, the newly synthesized double-stranded molecule is subjected to denaturing conditions using any of the procedures described above to provide single-stranded molecules.

The steps of denaturing, annealing, and extension product synthesis can be repeated as often as needed to amplify the target polymorphic locus nucleic acid sequence to the extent necessary for detection. The amount of the specific nucleic acid sequence produced will accumulate in an exponential fashion. Amplification is described in PCR. A Practical Approach, ILR Press, Eds. M. J. McPherson, P. Quirke, and G. R. Taylor, 1992.

The amplification products may be detected by Southern blots analysis, without using radioactive probes. In such a process, for example, a small sample of DNA containing a very low level of the nucleic acid sequence of the polymorphic locus is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the probe, or will be able to ascertain such, using routine experimentation.

Sequences amplified by the methods of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology,3:1008-1012, 1985), allele-specific oligonucleotide (ASO) probe analysis (Conner, et al., Proc. Natl. Acad. Sci. U.S.A., 80:278, 1983), oligonucleotide ligation assays (OLAs) (Landgren, et al., Science,241:1007, 1988), and the like. Molecular techniques for DNA analysis have been reviewed (Landgren, et al., Science,242:229-237, 1988).

Preferably, the method of amplifying is by PCR, as described herein and as is commonly used by those of ordinary skill in the art. Alternative methods of amplification have been described and can also be employed as long as the BRCA1 locus amplified by PCR using primers of the invention is similarly amplified by the alternative means. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 10⁸ copies within 60 to 90 minutes. Alternatively, nucleic acid can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, amplification is 10⁸ to 10⁹ fold. Another amplification system useful in the method of the invention is the Qβ Replicase System. The Qβ replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest. Another nucleic acid amplification technique, ligase chain reaction (LCR), works by using two differently labeled halves of a sequence of interest which are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligonucleotide probe pairs, and the RCR fills and joins the gap, mimicking DNA repair.

Nucleic acid amplification by strand displacement activation (SDA) utilizes a short primer containing a recognition site for hincII with short overhang on the 5′ end which binds to target DNA. A DNA polymerase fills in the part of the primer opposite the overhang with sulfur-containing adenine analogs. HincII is added but only cuts the unmodified DNA strand. A DNA polymerase that lacks 5′ exonuclease activity enters at the cite of the nick and begins to polymerize, displacing the initial primer strand downstream and building a new one which serves as more primer. SDA produces greater than 10⁷-fold amplification in 2 hours at 37° C. Unlike PCR and LCR, SDA does not require instrumented Temperature cycling.

Another method is a process for amplifying nucleic acid sequences from a DNA or RNA template which may be purified or may exist in a mixture of nucleic acids. The resulting nucleic acid sequences may be exact copies of the template, or may be modified. The process has advantages over PCR in that it increases the fidelity of copying a specific nucleic acid sequence, and it allows one to more efficiently detect a particular point mutation in a single assay. A target nucleic acid is amplified enzymatically while avoiding strand displacement. Three primers are used. A first primer is complementary to the first end of the target. A second primer is complementary to the second end of the target. A third primer which is similar to the first end of the target and which is substantially complementary to at least a portion of the first primer such that when the third primer is hybridized to the first primer, the position of the third primer complementary to the base at the 5′ end of the first primer contains a modification which substantially avoids strand displacement. This method is detailed in U.S. Pat. No. 5,593,840 to Bhatnagar et al. 1997. Although PCR is the preferred method of amplification if the invention, these other methods can also be used to amplify the BRCA1 locus as described in the method of the invention.

Finally, recent application of DNA chips or microarray technology where DNA or oligonucleotides are immobilized on small solid support may also be used to rapidly sequence sample gene and analyze its expression. Typically, high density arrays of DNA fragment are fabricated on glass or nylon substrates by in situ light-directed combinatorial synthesis or by conventional synthesis followed by immobilization (U.S. Pat. No. 5,445,934). Sample DNA or RNA may be amplified by PCR, labeled with a fluorescent tag, and hybridized to the microarray. Examples of this technology are provided in U.S. Pat. No. 5,510, 270 and U.S. Pat No. 5,547,839, incorporated herein by reference.

The BRCA1^{(omi)} DNA coding sequences were obtained by end to end sequencing of the BRCA1 alleles of five subjects in the manner described above followed by analysis of the data obtained. The data obtained provided us with the opportunity to evaluate seven previously published polymorphisms and to affirm or correct where necessary, the frequency of occurrence of alternative codons.

### Gene therapy

The coding sequences can be used for gene therapy. A variety of methods are known for gene transfer, any of which might be available for use.

### Direct injection of Recombinant DNA in vivo

1. Direct injection of “naked” DNA directly with a syringe and needle into a specific tissue, infused through a vascular bed, or transferred through a catheter into endothelial cells.

2. Direct injection of DNA that is contained in artificially generated lipid vesicles or other suitable encapsulating vehicle.

3. Direct injection of DNA conjugated to a target receptor structure, such as a diptheria toxin, an antibody or other suitable receptor.

4. Direct injection by particle bombardment, where the DNA is coated onto gold particles and shot into the cells.

### Human Artificial Chromosomes

This novel gene delivery approach involves the use of human chromosomes that have been striped down to contain only the essential components for replication and the genes desired for transfer.

### Receptor-Mediated Gene Transfer

DNA is linked to a targeting molecule that will bind to specific cell-surface receptors, inducing endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes.

### Recombinant Virus Vectors

Several vectors are used in gene therapy. Among them are the Moloney Murine Leukemia Virus (MoMLV) Vectors, the adenovirus vectors, the adeno-Associated Virus (AAV) vectors, the retrovirus vectors, the herpes simplex virus (HSV) vectors, the poxvirus vectors, and human immunodeficiency virus (HIV) vectors.

### Gene Replacement and Repair

The ideal genetic manipulation for treatment of a genetic disease would be the actual replacement of the defective gene with a normal copy of the gene. Homologous recombination is the term used for switching out a section of DNA and replacing it with a new piece. By this technique, the defective gene can be replaced with a normal gene which expresses a functioning BRCA1 tumor growth inhibitor protein. A complete description of gene therapy can also be found in “Gene Therapy A Primer For Physicians 2d Ed. by Kenneth W. Culver, M. D. Publ. Mary Ann Liebert Inc. (1996). Two Gene Therapy Protocols for BRCA1 are approved by the Recombinant DNA Advisory Committee for Jeffrey T. Holt et al. They are listed as 9602-148, and 9603-149 and are available from the NIH. The isolated BRCA1 gene can be synthesized or constructed from amplification products and inserted into a vector such as the LXSN vector.

### A BRCA1^{(OMI)} Polypeptide or its Functional Equivalent

It has been shown that active BRCA1 protein inhibits the growth of the cancer cells and reduces tumorigenesis. Thus, the growth of breast or ovarian cancer may be arrested or prevented by increasing the BRCA1 protein level where inadequate functional BRCA1 activity is responsible for breast or ovarian cancer. The cDNA and amino acid sequences of the BRCA1^{(omi1)}, BRCA1^{(omi2)} and BRCA1^{(omi3)} haplotype are disclosed herein (SEQ ID Nos:1-6). All or a fragment of BRCA1^{(omi)} protein may be used in therapeutic or prophylactic treatment of breast or ovarian cancer. Such a fragment may have a similar biological function as the native BRCA1^{(omi)} protein or may have a desired biological function as specified below. BRCA1^{(omi)} polypeptides or their functional equivalents including homologous and modified polypeptide sequences are also within the scope of the present invention. Changes in the native sequence may be advantageous in producing or using the BRCA1^{(omi)} derived polypeptide or functional equivalent suitable for therapeutic or prophylactic treatment of breast or ovarian cancer. For example, these changes may be desirable for producing resistance against in vivo proteolytic cleavage, for facilitating transportation and delivery of therapeutic reagents, for localizing and compartmentalizing tumor suppressing agents, or for expression, isolating and purifying the target species.

There are a variety of methods to produce an active BRCA1^{(omi)} polypeptide or a functional equivalent as a tumor growth inhibitor. For example, one or more amino acids may be substituted, deleted, or inserted using methods well known in the art (Maniatis et al., 1982). Considerations of polarity, charge, solubility, hydrophobicity, hydrophilicity and/or the amphiphathic nature of the amino acids play an important role in designing homologous polypeptide changes suitable for the intended treatment. In particular, conservative amino acid substitution using amino acids that are related in side-chain structure and charge may be employed to preserve the chemical and biological property. A homologous polypeptide typically contains at least 70% sequence homology to the native sequence. Unnatural forms of the polypeptide may also be incorporated so long as the modification retains substantial biological activity. These unnatural forms typically include structural mimics and chemical medications, which have similar three-dimensional structures as the active regions of the native BRCA1^{(omi)} protein. For example, these modifications may include terminal D-amino acids, cyclic peptides, unnatural amino acids side chains, pseudopeptide bonds, N-terminal acetylation, glycosylation, and biotinylation, etc. These unnatural forms polypeptide may have a desired biological function, for example, they be particularly robust in the presence of cellular or serum proteases and exopeptidases. An effective BRCA1^{(omi)} polypeptide or a functional equivalent may also be recognized by the reduction of the native BRCA1 protein. Regions of the BRCA1 protein may be systematically deleted to identify which regions are essential for tumor growth inhibitor activity. These smaller fragments of BRCA1^{(omi)} protein may then be subjected to structural and functional modification to derive the therapeutically or prophylactically effective regiments. Finally, drugs, natural products or small molecules may be screened or synthesized to mimic the function of the BRCA1 protein. Typically, the active species retain the essential three-dimensional shape and chemical reactivity, and therefore retain the desired aspects of the biological activity of the native BRCA1 protein. The activity and function of the BRCA1 protein may include transcriptional activation, granin, DNA repair, among others. Functions of BRCA1 protein are also reviewed in Bertwistle and Ashworth, Curr. Opin. Genet. Dev. 8(1): 14-20 (1998) and Zhang et al., Cell 92:433-436 (1998). It will be apparent to one skilled in the art that a BRCA1^{(omi)} polypeptide or a functional equivalent may be selected because such polypeptide or functional equivalent possesses similar biological activity as the native BRCA1 protein.

### Expression of the BRCA1^{(OMI)} Protein and Polypeptide in Host Cells

All or fragments of the BRCA1^{(omi)} protein and polypeptide may be produced by host cells that are capable of directing the replication and the expression of foreign genes. Suitable host cells include prokaryotes, yeast cells, or higher eukaryotic cells, which contain an expression vector comprising all or a fragment of BRCA1^{(omi)} cDNA sequence (SEQ. ID No:1, 3, or 5) operatively linked to one or more regulatory sequences to produce the intended BRCA1^{(omi)} protein or polypeptide. Prokaryotes may include gram negative or gram positive organisms, for example E. coli or Bacillus strains. Suitable eukaryotic host cells may include yeast, virus, and mamalian systems. For example, Sf9 insect cells and human cell lines, such as COS, MCF7, HeLa, 293T, HBL100, SW480, and HCT116 cells.

A broad variety of suitable expression vectors are available in the art. An expression vector typically contains an origin of replication, a promoter, a phenotypic selection gene (antibiotic resistance or autotrophic requirement), and a DNA sequence coding for all or fragments of the BRCA1^{(omi)} protein. The expression vectors may also include other operatively linked regulatory DNA sequences known in the art, for example, stability leader sequences, secretory leader sequences, restriction enzyme cleavage sequences, polyadenylation sequences, and termination sequences, among others. The essential and regulatory elements of the expression vector must be compatible with the intended host cell. Suitable expression vectors containing the desired coding and control regions may be constructed using standard recombinant DNA techniques known in the art, many of which are described in Sambrook, et al., Molecular Cloning: A Laboratory Manual, Second Edition, Cold Spring Harbor Laboratory, Cold Spring Harbor, N.Y. (1989). For example, suitable origins of replication may include Col E1, SV40 viral and M13 origins of replication. Suitable promoters may be constitutive or inducible, for example, tac promoter, lac Z promoter, SV40 promoter, MMTV promoter, and LXSN promoter. Examples of selectable markers include neomycin, ampicillin, and hygromycin resistance and the like. Many suitable prokaryotic, viral and mammalian expression vectors may be obtained commercially, for example, from Invitrogen Corp., San Diego, Calif. or from Clontech, Palo Alto, Calif. It may be desirable that the BRCA1^{(omi)} protein or polypeptide is produced as a fusion protein to enhance the expression in selected host cells, to detect the expression in transfected cells, or to simplify the purification process. Suitable fusion partners for the BRCA^{(omi)} protein or polypeptide are well known in the art and may include β-galactosidase, glutathione-S-transferase and poly-histidine tag.

Expression vectors may be introduced into host cells by various methods known in the art. The transformation procedure used depends upon the host to be transformed. Methods for introduction of vectors into host cells may include calcium phosphate precipitation, electrosporation, dextran-mediated transfection, liposome encapsulation, nucleus microinjection, and viral or phage infection, among others.

Once an expression vector has been introduced into a suitable host cell, the host cell may be cultured under conditions permitting expression of large amounts of the BRCA1^{(omi)} protein or polypeptide. The expression product may be identified by many approaches well known in the art, for example, sequencing after PCR-based amplification, hybridization using probes complementary to the desired DNA sequence, the presence or absence of marker gene functions such as enzyme activity or antibiotic resistance, the level of mRNA production encoding the intended sequence, immunological detection of a gene product using monoclonal and polyclonal antibodies, such as Western blotting or ELISA. The BRCA1^{(omi)} protein or polypeptides produced in this manner may then be isolated following cell lysis and purified using various protein purification techniques known in the art, for example, ion exchange chromatography, gel filtration chromatography and immunoaffinity chromatography.

It is generally preferred that whenever possible, longer fragments of BRCA1^{(omi)} protein or polypeptide are used, particularly to include the desired functional domains of BRCA1 protein. Expression of shorter fragments of DNA may be useful in generating BRCA1^{(omi)} derived immunogen for the production of anti-BRCA1^{(omi)} antibodies. It should, of course, be understood that not all expression vectors, DNA regulatory sequences or host cells will function equally well to express the BRCA1^{(omi)} protein or polypeptides of the present invention. However, one of ordinary skill in the art may make a selection among expression vectors, DNA regulatory sequences, host cells, and codon usage in order to optimize expression using known technology in the art without undue experimentation. Studies of the BRCA1 protein and examples of genetic manipulation of the BRAC1 protein are summarized in two recent review articles, Bertwistle and Ashworth, Curr. Opin. Genet. Dev. 8(1): 14-20 (1998) and Zhang et al., Cell 92:433-436 (1998).

### In Vitro Synthesis and Chemical Synthesis

Although it is preferred that the BRCA1^{(omi)} protein or polypeptides be obtained by overexpression in prokaryotic or eukaryotic host cells, the BRCA1^{(omi)} polypeptides or their functional equivalents may also be obtained by in vitro translation or synthetic means by methods known to those of ordinary skill in the art. For example, in vitro translation may employ a mRNA encoded by a DNA sequence coding for all or fragments of the BRCA1^{(omi)} protein or polypeptides. Chemical synthesis methodology such as solid phase synthesis may be used to synthesize a BRCA1^{(omi)} polypeptide structural mimic and chemically modified analogs thereof. The polypeptides or the modifications and mimic thereof produced in this manner may then be isolated and purified using various purification techniques, such as chromatographic procedures including ion exchange chromatography, gel filtration chromatography and immunoaffinity chromatography.

### Protein Replacement Therapy

The ability of the BRCA1 protein to inhibit tumor growth demonstrates that various BRCA1 protein targeted therapies may be utilized in treating and preventing tumors in breast and ovarian cancer. The present invention therefore includes therapeutic and prophylactic treatment of breast and ovarian cancer using therapeutic pharmaceutical compositions containing the BRCA1^{(omi)} protein, polypeptides, or their functional equivalents. For example, protein replacement therapy may involve directly administering the BRCA1^{(omi)} protein, a BRCA1^{(omi)} polypeptide, or a functional equivalent in a pharmaceutically effective carrier. Alternatively, protein replacement therapy may utilize tumor antigen specific antibody fused to the BRCA1^{(omi)} protein, a polypeptide, or a functional equivalent to deliver anti-cancer regiments specifically to the tumor cells.

To prepare the pharmaceutical compositions of the present invention, an active BRCA1^{(omi)} protein, a polypeptide, or its functional equivalent is combined with a pharmaceutical carrier selected and prepared according to conventional pharmaceutical compounding techniques. A suitable amount of the composition may be administered locally to the site of a tumor or systemically to arrest the proliferation of tumor cells. The methods for administration may include parenteral, oral, or intravenous, among others according to established protocols in the art.

Pharmaceutically acceptable solid or liquid carriers or components which may be added to enhance or stabilize the composition, or to facilitate preparation of the composition include, without limitation, syrup, water, isotonic solution, 5% glucose in water or buffered sodium or ammonium acetate solution, oils, glycerin, alcohols, flavoring agents, preservatives, coloring agents, starches, sugars, diluents, granulating agents, lubricants, binders, and sustained release materials. The dosage at which the therapeutic compositions are administered may vary within a wide range and depends on various factors, such as the stage of cancer progression, the age and condition of the patient, and may be individually adjusted.

### Diagnostic Reagents

The BRCA1^{(omi)} protein, polypeptides, their functional equivalents, antibodies, and polynucleotides may be used in a wide variety of ways in addition to gene therapy and protein replacement therapy. They may be useful as diagnostic reagents to measure normal or abnormal activity of BRCA1 at the DNA, RNA, and protein level. The present invention therefore encompasses the diagnostic reagents derived from the BRCA1^{(omi)} cDNA and protein sequences as set forth in SEQ. ID. Nos:1-6. These reagents may be utilized in methods for monitoring disease progression, for determining patients suited for gene and protein replacement therapy, or for detecting the presence or quantifying the amount of a tumor growth inhibitor following such therapy. Such methods may involve conventional histochemical techniques, such as obtaining a tumor tissue from the patient, preparing an extract and testing this extract for tumor growth or metabolism. For example, the test for tumor growth may involve measuring abnormal BRCA1^{(omi)} activity using conventional diagnostic assays, such as Southern, Northern, and Western blotting, PCR, RT-PCR, immunoassay, and immunoprecipitation. In biopsies of tumor tissues, the loss of BRCA1^{(omi)} expression in tumor tissue may be verified by RT-PCR and Northern blotting at the RNA level. A Southern blot analysis, genomic PCR, or fluorescence in situ hybridization (FISH) may also be performed to examine the mutations of BRCA1 at the DNA level. And, a Western blotting, protein truncation assay, or immunoprecipitation may be utilized to analysis the effect at the protein level.

These diagnostic reagents are typically either covalently or non convalently attached to a detectable label. Such a label includes a radioactive label, a colorimetric enzyme label, a fluorescence label, or an epitope label. Frequently, a reporter gene downstream of the regulatory sequences is fused with the BRCA1^{(omi)} protein or polypeptide to facilitate the detection and purification of the target species. Commonly used reporter genes in BRCA1 fusion proteins include β-galactosidase and luciferase gene.

The BRCA1^{(omi)} protein, polypeptides, their functional equivalents, antibodies, and polynucleotides may also be useful in the study of the characteristics of the BRCA1 protein, such as structure and function of BRCA1 in oncogenesis or subcellular localization of BRCA1 protein in normal and cancerous cells. For example, yeast two-hybrid system has been frequently used in the study of cellular function of BRCA1 to identify the regulator and effector of BRCA1 growth control pathways (See reviews of Bertwistle and Ashworth, 1998 and Zhange et al., 1998). In addition, the BRCA1^{(omi)} protein, polypeptides, their functional equivalents, antibodies, and polynucleotides may also be used in in vivo cell based and in vitro cell free assays to screen natural products and synthetic compounds which may mimic, regulate or stimulate BRCA1 protein function.

### Antisense Inhibition

Antisense suppression of endogenous BRCA1 expression may assess the effect of the BRCA1 protein on cell growth inhibition using known method in the art (Crooke, Annu. Rev. Pharmacol. Toxicol. 32:329-376 (1992) and Robinson-Benion and Holt, Methods Enzymol. 254:363-375 (1995)). Given the cDNA sequence as set forth in SEQ ID. NO:1, one of skill in the art can readily obtain anti-sense strand of DNA and RNA sequences to interfere with the production of the wild-type BRCA1^{(omi)} protein or the mutated form of BRCA1 protein. Alternatively, antisense oligonucleotide may be designed to target the control sequences of BRCA1^{(omi)} gene to reduce or prevent the expression of the endogenous BRCA1^{(omi)} gene. Examples of using oligonucleotide-based antisense technology to inhibit the BRCA1 expression are provided in Husain et al., Cancer Res. 58:1120-1123 (1998).

### Antibodies

The BRCA1^{(omi)} protein, polypeptides, or their functional equivalent may be used as immunogens to prepare polyclonal or monoclonal antibodies capable of binding the BRCA1 derived antigens in a known manner (Harlow & Lane, Antibodies: A Laboratory Manual, Cold Spring Harbor Laboratory, Cold Spring Harbor, N.Y., 1988). These antibodies may be used for the detection of the BRCA1 protein, polypeptides, or a functional equivalent in an immunoassay, such as ELISA, Western blot, radioimmunoassay, enzyme immunoassay, and immunocytochemistry. Typically, an anti-BRCA1^{(omi)} antibody is in solution or is attached to a solid surface such as a plate, a particle, a bead, or a tube. The antibody is allowed to contact a biological sample or a blot suspected of containing the BRCA1 protein or polypeptide to form a primary immunocomplex. After sufficient incubation period, the primary immunocomplex is washed to remove any non-specifically bound species. The amount of specifically bound BRCA1 protein or polypeptide may be determined using the detection of an attached label or a marker, such as a radioactive, a fluorescent, or an enzymatic label. Alternatively, the detection of BRCA1 derived antigen is allowed by forming a secondary immunocomplex using a second antibody which is attached with a such label or marker. The antibodies may also be used in affinity chromatography for isolating or purifying the BRCA1 protein, polypeptides or their functional equivalents. Examples of preparing and using anti-BRCA1 antibodies are provided in Ruffner et al., Proc. Natl. Acad. Sci. USA 94:7138-7143 (1997) and Jensen et al., Nat. Genetics 12:303-308 (1996).

### Example 1

### Determination of the Coding Sequence of a BRCA1^{(omi)} Gene From Five Individuals

### Materials and Methods

Approximately 150 volunteers were screened in order to identify individuals with no cancer history in their immediate family (i.e. first and second degree relatives). Each person was asked to fill out a hereditary cancer prescreening questionnaire See TABLE I below. Five of these were randomly chosen for end-to-end sequencing of their BRCA1 gene. A first degree relative is a parent, sibling, or offspring. A second degree relative is an aunt, uncle, grandparent, grandchild, niece, nephew, or half-sibling. Genomic DNA was isolated from white blood cells of five subjects selected from analysis of their answers to the questions above. Dideoxy sequence analysis was performed following polymerase chain reaction amplification.

All exons of the BRCA1 gene were subjected to direct dideoxy sequence analysis by asymmetric amplification using the polymerase chain reaction (PCR) to generate a single stranded product amplified from this DNA sample. Shuldiner, et al., Handbook of Techniques in Endocrine Research, p. 457-486, DePablo, F., Scanes, C., eds., Academic Press, Inc., 1993. Fluorescent dye was attached for automated sequencing using the Taq Dye Terminator® Kit (Perkin-Elmer cat# 401628). DNA sequencing was performed in both forward and reverse directions on an Applied Biosystems, Inc. (ABI) automated Model 377® sequencer. The software used for analysis of the resulting data was Sequence Navigator® software purchased through ABI.

1. Polymerase Chain Reaction (PCR) Amplification

Genomic DNA (100 nanograms) extracted from white blood cells of five subjects. Each of the five samples was sequenced end to end. Each sample was amplified in a final volume of 25 microliters containing 1 microliter (100 nanograms) genomic DNA, 2.5 microliters 10×PCR buffer (100 mM Tris, pH 8.3, 500 mM KCl, 1.2 mM MgCl₂), 2.5 microliters 10×DNTP mix (2 mM each nucleotide), 2.5 microliters forward primer, 2.5 microliters reverse primer, and 1 microliter Taq polymerase (5 units), and 13 microliters of water.

The primers in Table II were used to carry out amplification of the various sections of the BRCA1 gene samples. The primers were synthesized on an DNA/RNA Model 394® Synthesizer. Thirty-five cycles were performed, each consisting of denaturing (95° C.; 30 seconds), annealing (55° C.; 1 minute), and extension (72° C.; 90 seconds), except during the first cycle in which the denaturing time was increased to 5 minutes, and during the last cycle in which the extension time was increased to 5 minutes.

PCR products were purified using QIA-Quick® PCR purification kits (Qiagen Cat# 28104; Chatsworth, Calif.). Yield and purity of the PCR product determined spectrophotometrically at OD₂₆₀ on a Beckman DU 650 spectrophotometer.

2. Dideoxy Sequence Analysis

Fluorescent dye was attached to PCR products for automated sequencing using the Taq Dye Terminator® kit (Perkin-Elmer cat# 401628). DNA sequencing was performed in both forward and reverse directions on an Applied Biosystems, Inc. (ABI), Foster City, Calif., Automated Model 377® sequencer. The software used for analysis of the resulting data was “Sequence Navigator® Software” purchased through ABI.

TABLE I
Hereditary Cancer Pre-Screening Questionnaire

Part A: Answer the following questions about your family

| | |
|---|---|
| 1. | To your knowledge, has anyone in your family been diagnosed with a very specific hereditary |
| | colon disease called Familial Adenomatous Polyposis (FAP)? |
| 2. | To your knowledge, have you or any aunt had breast cancer diagnosed before the age 35? |
| 3. | Have you had Inflammatory Bowel Disease, also called Crohn’s Disease or Ulcerative Colitis, for |
| | more than 7 years? |

Part B: Refer to the list of cancers below for your responses only to questions in Part B

| | | |
|---|---|---|
| Bladder Cancer | Lung Cancer | Pancreatic Cancer |
| Breast Cancer | Gastric Cancer | Prostate Cancer |
| Colon Cancer | Malignant Melanoma | Renal Cancer |
| Endometrial Cancer | Ovarian Cancer | Thyroid Cancer |

| | |
|---|---|
| 4. | Have your mother or father, your sisters or brothers or your children had any of the listed |
| | cancers? |
| 5 | Have there been diagnosed in your mother’s brothers or sisters, or your mother’s parents more than one |
| | of the cancers in the above list? |
| 6. | Have there been diagnosed in your father’s brothers or sisters, or your father’s parents more than one of |
| | the cancers in the above list? |

Part C: Refer to the list of relatives below for responses only to questions in Part C

| | |
|---|---|
| You | Your mother |
| Your sisters or brothers | Your mothers’s sisters or brothers (maternal aunts and uncles) |
| Your children | Your mother’s parents (maternal grandparents) |

| | |
|---|---|
| 7 | Have there been diagnosed in these relatives 2 or more identical types of cancer? |
| | Do not count “simple” skin cancer, also called basal cell or squamous cell skin cancer. |
| 8. | Is there a total of 4 or more of any cancers in the list of relatives above other than “simple” skin |
| | cancers? |

Part D: Refer to the list of relatives below for responses only to questions in Part D.

| | |
|---|---|
| You | Your father |
| Your sisters or brothers | Your fathers’s sisters or brothers (paternal aunts and uncles) |
| Your children | Your father’s parents (paternal grandparents) |

| | |
|---|---|
| 9. | Have there been diagnosed in these relatives 2 or more identical types of cancer? |
| | Do not count “simple” skin cancer, also called basal cell or squamous cell skin cancer. |
| 10. | Is there a total of 4 or more of any cancers in the list of relatives above other than “simple” skin |
| | cancers? |

© Copyright 1996, OncorMed, Inc.

TABLE II
BRCA1 PRIMERS AND SEQUENCING DATA

| | | | SEQ. | PRIMER | | EXON |
|---|---|---|---|---|---|---|
| EXON | LABEL | SEQUENCE | ID NO. | SIZE | Mg⁺⁺ | SIZE |
| 2 | 2F | 5′-GAA GTT GTC ATT TTA TAA ACC TTT-3′ | 7 | 24 | 1.6 | ˜275 |
| | 2R | 5′-TGT CTT TTC TTC CCT AGT ATG T-3′ | 8 | 22 | | |
| 3 | 3F | 5′-TCC TGA CAC AGC AGA CAT TTA-3′ | 9 | 21 | 1.4 | ˜375 |
| | 3R | 5′-TTG GAT TTT CGT TCT CAC TTA-3′ | 10 | 21 | | |
| 5 | 5F | 5′-CTC TTA AGG GCA GTT GTG AG-3′ | 11 | 20 | 1.2 | ˜275 |
| | 5R | 5′-TTC CTA CTG TGG TTG CTT CC-3′ | 12 | 20¹ | | |
| 6 | 6/7F | 5′-CTT ATT TTA GTG TCC TTA AAA GG-3′ | 13 | 23 | 1.6 | ˜250 |
| | 6R | 5′-TTT CAT GGA CAG CAC TTG AGT G-3′ | 14 | 22 | | |
| 7 | 7F | 5′-CAC AAC AAA GAG CAT ACA TAG GG-3′ | 15 | 23 | 1.6 | ˜275 |
| | 6/7R | 5′-TCG GGT TCA CTC TGT AGA AG-3′ | 16 | 20 | | |
| 8 | 8F1 | 5′-TTC TCT TCA GGA GGA AAA GCA-3′ | 17 | 21 | 1.2 | ˜270 |
| | 8R1 | 5′-GCT GCC TAC CAC AAA TAC AAA-3′ | 18 | 21 | | |
| 9 | 9F | 5′-CCA CAG TAG ATG CTC AGT AAA TA-3′ | 19 | 23 | 1.2 | ˜250 |
| | 9R | 5′-TAG GAA AAT ACC AGC TTC ATA GA-3′ | 20 | 23 | | |
| 10 | 10F | 5′-TGG TCA GCT TTC TGT AAT CG-3′ | 21 | 20 | 1.6 | ˜250 |
| | 10R | 5′-GTA TCT ACC CAC TCT CTT CTT CAG-3′ | 22 | 24 | | |
| 11A | 11AF | 5′-CCA CCT CCA AGG TGT ATC A-3′ | 23 | 19 | 1.2 | 372 |
| | 11AR | 5′-TGT TAT GTT GGC TCC TTG CT-3′ | 24 | 20 | | |
| 11B | 11BF1 | 5′-CAC TAA AGA CAG AAT GAA TCT A-3 | 25 | 21 | 1.2 | ˜400 |
| | 11BR1 | 5′-GAA GAA CCA GAA TAT TCA TCT A-3′ | 26 | 21 | | |
| 11C | 11CF1 | 5′-TGA TGG GGA GTC TGA ATC AA-3′ | 27 | 20 | 1.2 | ˜400 |
| | 11CR1 | 5′-TCT GCT TTC TTG ATA AAA TCC T-3′ | 28 | 22 | | |
| 11D | 11DF1 | 5′-AGC GTC CCC TCA CAA ATA AA-3′ | 29 | 20 | 1.2 | ˜400 |
| | 11DR1 | 5′-TCA AGC GCA TGA ATA TGC CT-3′ | 30 | 20 | | |
| 11E | 11EF | 5′-GTA TAA GCA ATA TGG AAC TCG A-3′ | 31 | 22 | 1.2 | 388 |
| | 11ER | 5′-TTA AGT TCA CTG GTA TTT GAA CA-3′ | 32 | 223 | | |
| 11F | 11FF | 5′-GAC AGC GAT ACT TTC CCA GA-3′ | 33 | 20 | 1.2 | 382 |
| | 11FR | 5′-TGG AAC AAC CAT GAA TTA GTC-3′ | 34 | 21 | | |
| 11G | 11GF | 5′-GGA AGT TAG CAC TCT AGG GA-3′ | 35 | 29 | 1.2 | 423 |
| | 11GR | 5′-GCA GTG ATA TTA ACT GTC TGT A-3′ | 36 | 22 | | |
| 11H | 11HF | 5′-TGG GTC CTT AAA GAA ACA AAGT-3′ | 37 | 22 | 1.2 | 366 |
| | 11HR | 5′-TCA GGT GAC ATT GAA TCT TCC-3′ | 38 | 21 | | |
| 11I | 11IF | 5′-CCA CTT TTT CCC ATC AAG TCA-3′ | 39 | 21 | 1.2 | 377 |
| | 11IR | 5′-TCA GGA TGC TTA CAA TTA CTT C-3′ | 40 | 21 | | |
| 11J | 11JF | 5′-CAA AAT TGA ATG CTA TGC TTA GA-3′ | 41 | 23 | 1.2 | 377 |
| | 11JR | 5′-TCG GTA ACC CTG AGC CAA AT-3′ | 42 | 20 | | |
| 11K | 11KF | 5′-GCA AAA GCG TCC AGA AAG GA-3′ | 43 | 20 | 1.2 | 396 |
| | 11KR-1 | 5′-TAT TTG CAG TCA AGT CTT CCA A-3′ | 44 | 22 | | |
| 11L | 11LF-1 | 5′-GTA ATA TTG GCA AAG GCA TCT-3′ | 45 | 22 | 1.2 | 360 |
| | 11LR | 5′-TAA AAT GTG CTC CCC AAA AGC A-3′ | 46 | 22 | | |
| 12 | 12F | 5′-GTC CTG CCA ATG AGA AGA AA-3′ | 47 | 20 | 1.2 | ˜300 |
| | 12R | 5′-TGT CAG CAA ACC TAA GAA TGT-3′ | 48 | 21 | | |
| 13 | 13F | 5′-AAT GGA AAG CTT CTC AAA GTA-3′ | 49 | 21 | 1.2 | ˜325 |
| | 13R | 5′-ATG TTG GAG CTA GGT CCT TAC-3′ | 50 | 21 | | |
| 14 | 14F | 5′-CTA ACC TGA ATT ATC ACT ATC A-3′ | 51 | 22 | 1.2 | ˜310 |
| | 14R | 5′-GTG TAT AAA TGC CTG TAT GCA-3′ | 52 | 21 | | |
| 15 | 15F | 5′-TGG CTG CCC AGG AAG TAT G-3′ | 53 | 19 | 1.2 | ˜375 |
| | 15R | 5′-AAC CAG AAT ATC TTT ATG TAG GA-3′ | 54 | 23 | | |
| 16 | 16F | 5′-AAT TCT TAA CAG AGA CCA GAA C-3′ | 55 | 22 | 1.6 | ˜550 |
| | 16R | 5′-AAA ACT CTT TCC AGA ATG TTG T-3′ | 56 | 22 | | |
| 17 | 17F | 5′-GTG TAG AAC GTG CAG GAT TG-3′ | 57 | 20 | 1.2 | ˜275 |
| | 17R | 5′-TCG CCT CAT GTG GTT TTA-3′ | 58 | 18 | | |
| 18 | 18F | 5′-GGC TCT TTA GCT TCT TAG GAC-3′ | 59 | 21 | 1.2 | ˜350 |
| | 18R | 5′-GAG ACC ATT TTC CCA GCA TC-3′ | 60 | 20 | | |
| 19 | 19F | 5′-CTG TCA TTC TTC CTG TGC TC-3′ | 61 | 20 | 1.2 | ˜250 |
| | 19R | 5′-CAT TGT TAA GGA AAG TGG TGC-3′ | 62 | 21 | | |
| 20 | 20F | 5′-ATA TGA CGT GTC TGC TCC AC-3′ | 63 | 20 | 1.2 | ˜425 |
| | 20R | 5′-GGG AAT CCA AAT TAC ACA GC-3′ | 64 | 20 | | |
| 21 | 21F | 5′-AAG CTC TTC CTT TTT GAA AGT C-3′ | 65 | 22 | 1.6 | ˜300 |
| | 21R | 5′-GTA GAG AAA TAG AAT AGC CTC T-3′ | 66 | 22 | | |
| 22 | 22F | 5′-TCC CAT TGA GAG GTC TTG CT-3′ | 67 | 20 | 1.6 | ˜300 |
| | 22R | 5′-GAG AAG ACT TCT GAG GCT AC-3′ | 68 | 20 | | |
| 23 | 23F-1 | 5′-TGA AGT GAC AGT TCC AGT AGT-3′ | 69 | 21 | 1.2 | ˜250 |
| | 23R-1 | 5′-CAT TTT AGC CAT TCA TTC AAC AA-3′ | 70 | 23 | | |
| 24 | 24F | 5′-ATG AAT TGA CAC TAA TCT CTG C-3′ | 71 | 22 | 1.4 | ˜285 |
| | 24R | 5′-GTA GCC AGG ACA GTA GAA GGA-3′ | 72 | 21 | | |

¹M-13 tagged

3. Results

Differences in the nucleic acids of the ten alleles from five individuals were found in seven locations on the gene. The changes and their positions are found on Table III, below.

at position 2201, AGC and AGT occur at frequencies from about 35-45%, and from about 55-65%, respectively;

at position 2430, TTG and CTG occur at frequencies from about 35-45%, and from about 55-65%, respectively;

at position 2731, CCG and CTG occur at frequencies from about 25-35%, and from about 65-75%, respectively;

at position 3232, GAA and GGA occur at frequencies from about 35-45%, and from about 55-65%, respectively;

at position 3667, AAA and AGA occur at frequencies from about 35-45%, and from about 55-65%, respectively;

at position 4427, TCT and TCC occur at frequencies from about 45-55%, and from about 45-55%, respectively; and

at position 4956, AGT and GGT occur at frequencies from about 35-45%, and from about 55-65%, respectively.

The data show that for each of the samples. The BRCA1 gene is identical except in the region of seven polymorphisms. These polymorphic regions, together with their locations, the amino acid groups of each codon, the frequency of their occurrence and the amino acid coded for by each codon are found in TABLE IV below.

TABLE III
PANEL TYPING

| ACID | | | |
|---|---|---|---|
| AMINO | NUCLEOTIDE | | |
| CHANGE | CHANGE | 1 2 3 4 5 | FREQUENCY |
| SER(SER) | 11E | C/C C/T C/T T/T T/T | 0.4C 0.6T |
| (694) | | | |
| LEU(LEU) | 11F | T/T C/T C/T C/C C/C | 0.4T 0.6C |
| (771) | | | |
| PRO(LEU) | 11G | C/T C/T C/T T/T T/T | 0.3C 0.7T |
| (871) | | | |
| GLU(GLY) | 11I | A/A A/G A/G G/G G/G | 0.4A 0.6G |
| (1038) | | | |
| LYS(ARG) | 11J | A/A A/G A/G G/G G/G | 0.4A 0.6G |
| (1183) | | | |
| SER(SER) | 13 | T/T T/T T/C C/C C/C | 0.5T 0.5C |
| (1436) | | | |
| SER(GLY) | 16 | A/A A/G A/G G/G G/G | 0.4A 0.6G |
| (1613) | | | |

TABLE IV
CODON AND BASE CHANGES IN SEVEN POLYMORPHIC SITES OF PRCA1 GENE

| | | | | | | | FREQUENCY |
|---|---|---|---|---|---|---|---|
| SAMPLE | BASE | POSITION | | CODON | | PUBLISHED | IN THIS |
| NAME | CHANGE | nt/aa | EXON | CHANGE | AA CHANGE | FREQUENCY² | STUDY |
| 2, 3, 4, 5 | C-T | 2201/694 | 11E | AGC(AGT) | SER-SER | UNPUBLISHED | C = 40% |
| 2, 3, 4, 5 | T-C | 2430/771 | 11F | TTG(CTG) | LEU-LEU | T = 67%¹³ | T = 40% |
| 1, 2, 3, 4, 5 | C-T | 2731/871 | 11G | CCG(CTG) | PRO-LEU | C = 34%¹² | C = 30% |
| 2, 3, 4, 5 | A-G | 3232/1038 | 11I | GAA(GGA) | GLU-GLY | A = 67%¹³ | A = 40% |
| 2, 3, 4, 5 | A-G | 3667/1183 | 11J | AAA(AGA) | LYS-ARG | A = 68%¹² | A = 40% |
| 3, 4, 5 | T-C | 4427/1436 | 13 | TCT(TCC) | SER-SER | T = 67%¹² | T = 50% |
| 2, 3, 4, 5 | A-G | 4956/1613 | 16 | AGT(GGT) | SER-GLY | A = 67%¹² | A = 40% |

²Reference numbers correspond to the Table of References below

### Example 2

### Determination of a Individual using BRCA1^{(omi)} and the Seven Polymorphisms for Reference

A person skilled in the art of genetic susceptibility testing will find the present invention useful for:

a) identifying individuals having a BRCA1 gene, who are therefore have no elevated genetic susceptibility to breast or ovarian cancer from a BRCA1 mutation;

b) avoiding misinterpretation of polymorphisms found in the BRCA1 gene;

Sequencing is carried out as in EXAMPLE 1 using a blood sample from the patient in question. However, a BRCA1 ^{(omi)} sequence is used for reference and the polymorphic sites are compared to the nucleic acid sequences listed above for codons at each polymorphic site. A sample is one which compares to a BRCA1^{(omi)} sequence and contains one of the base variations which occur at each of the polymorphic sites. The codons which occur at each of the polymorphic sites are paired here reference.

AGC and AGT at position 2201,

TTG and CTG at position 2430,.

CCG and CTG at position 2731,

GAA and GGA at position 3232,

AAA and AGA at position 3667,

TCT and TCC at position 4427, and

AGT and GGT at position 4956.

The availability of these polymorphic pairs provides added assurance that one skilled in the art can correctly interpret the polymorphic variations without mistaking a variation for a mutation.

Exon 11 of the BRCA1 gene is subjected to direct dideoxy sequence analysis by asymmetric amplification using the polymerase chain reaction (PCR) to generate a single stranded product amplified from this DNA sample. Shuldiner, et al., Handbook of Techniques in Endocrine Research, p. 457-486, DePablo, F., Scanes, C., eds., Academic Press, Inc., 1993. Fluorescent dye is attached for automated sequencing using the Taq Dye Terminator® Kit (Perkin-Elmer cat# 401628). DNA sequencing is performed in both forward and reverse directions on an Applied Biosystems, Inc. (ABI) automated Model 377® sequencer. The software used for analysis of the resulting data is “Sequence Navigator® software” purchased through ABI.

1. Polymerase Chain Reaction (PCR) Amplification

Genomic DNA (100 nanograms) extracted from white blood cells of the subject is amplified in a final volume of 25 microliters containing 1 microliter (100 nanograms) genomic DNA, 2.5 microliters 10×PCR buffer (100 mM Tris, pH 8.3, 500 mM KCl, 1.2 mM MgCl₂), 2.5 microliters 10×dNTP mix (2 mM each nucleotide), 2.5 microliters forward primer (BRCA1-11K-F, 10 micromolar solution), 2.5 microliters reverse primer (BRCA1-11K-R, 10 micromolar solution), and 1 microliter Taq polymerase (5 units), and 13 microliters of water.

The PCR primers used to amplify a patient's sample BRCA1 gene are listed in Table II. The primers were synthesized on an DNA/RNA Model 394® Synthesizer. Thirty-five cycles are of amplification are performed, each consisting of denaturing (95%C; 30 seconds), annealing (55%C; 1 minute), and extension (72° C.; 90 seconds), except during the first cycle in which the denaturing time is increased to 5 minutes, and during the last cycle in which the extension time is increased to 5 minutes.

PCR products are purified using Qia-quick® PCR purification kits (Qiagen, cat# 28104; Chatsworth, Calif.). Yield and purity of the PCR product determined spectrophotometrically at OD₂₆₀ on a Beckman DU 650 spectrophotometer.

2. Dideoxy Sequence Analysis

Fluorescent dye is attached to PCR products for automated sequencing using the Taq Dye Terminator® Kit (Perkin-Elmer cat# 401628). DNA sequencing is performed in both forward and reverse directions on an Applied Biosystems, Inc. (ABI) Foster City, Calif.., automated Model 377® sequencer. The software used for analysis of the resulting data is “Sequence Navigator® software” purchased through ABI. The BRCA1^{(omi1)} SEQ. ID. NO.:1 sequence is entered into the Sequence Navigator® software as the Standard for comparison. The Sequence Navigator® software compares the sample sequence to the BRCA1^{(omi1)} SEQ. ID. NO.:1 standard, base by base. The Sequence Navigator® software highlights all differences between the BRCA1^{(omi1)} SEQ. ID. NO.:1 DNA sequence and the patient's sample sequence.

A first technologist checks the computerized results by comparing visually the BRCA1^{(omi1)} SEQ. ID. NO.:1 standard against the patient's sample, and again highlights any differences between the standard and the sample. The first primary technologist then interprets the sequence variations at each position along the sequence. Chromatograms from each sequence variation are generated by the Sequence Navigator® software and printed on a color printer. The peaks are interpreted by the first primary technologist and a second primary technologist. A secondary technologist then reviews the chromatograms. The results are finally interpreted by a geneticist. In each instance, a variation is compared to known polymorphisms for position and base change. If the sample BRCA1 sequence matches the BRCA1^{(omi1)} SEQ. ID. NO.:1 standard, with only variations within the known list of polymorphisms, it is interpreted as a gene sequence.

### Example 3

### Determining the Absence of a Mutation in the BRCA1 Gene using BRCA1^{(omi1)} and Seven Polymorphisms for Reference

A person skilled in the art of genetic susceptibility testing will find the present invention useful for determining the presence of a known or previously unknown mutation in the BRCA1 gene. A list of mutations of BRCA1 is publicly available in the Breast Cancer Information Core at:

http://www.nchgr.nih.gov/dir/lab_transfer/Dic. This data site became publicly available on Nov. 1, 1995. Friend, S. et al. Nature Genetics 11:238, (1995).

Sequencing is carried out as in EXAMPLE 1 using a blood sample from the patient in question. However, a BRCA1^{(omi)} sequence is used for reference and polymorphic sites are compared to the nucleic acid sequences listed above for codons at each polymorphic site. A sample is one which compares to the BRCA1^{(omi2)} SEQ. ID. NO.:3 sequence and contains one of the base variations which occur at each of the polymorphic sites. The codons which occur at each of the polymorphic sites are paired here reference.

AGC and AGT at position 2201,

TTG and CTG at position 2430,

CCG and CTG at position 2731,

GAA and GGA at position 3232,

AAA and AGA at position 3667,

TCT and TCC at position 4427, and

AGT and GGT at position 4956.

The availability of these polymorphic pairs provides added assurance that one skilled in the art can correctly interpret the polymorphic variations without mistaking a variation for a mutation.

Exon 11 of the BRCA1 gene is subjected to direct dideoxy sequence analysis by asymmetric amplification using the polymerase chain reaction (PCR) to generate a single stranded product amplified from this DNA sample. Shuldiner, et al., Handbook of Techniques in Endocrine Research, p. 457-486, DePablo, F., Scanes, C., eds., Academic Press, Inc., 1993. Fluorescent dye is attached for automated sequencing using the Taq Dye Terminator® Kit (Perkin-Elmer cat# 401628). DNA sequencing is performed in both forward and reverse directions on an Applied Biosystems, Inc. (ABI) automated Model 377® sequencer. The software used for analysis of the resulting data is “Sequence Navigator® software” purchased through ABI.

1. Polymerase Chain Reaction (PCR) Amplification

Genomic DNA (100 nanograms) extracted from white blood cells of the subject is amplified in a final volume of 25 microliters containing 1 microliter (100 nanograms) genomic DNA, 2.5 microliters 10×PCR buffer (100 mM Tris, pH 8.3, 500 mM KCl, 1.2 mM MgCl₂), 2.5 microliters 10×dNTP mix (2 mM each nucleotide), 2.5 microliters forward primer (BRCAL-1-11K-F, 10 micromolar solution), 2.5 microliters reverse primer (BRCA1-11K-R, 10 micromolar solution),and 1 microliter Taq polymerase (5 units), and 13 microliters of water.

The PCR primers used to amplify a patient's sample BRCA1 gene are listed in Table II. The primers were synthesized on an DNA/RNA Model 394® Synthesizer. Thirty-five cycles are of amplification are performed, each consisting of denaturing (95%C; 30 seconds), annealing (55%C; 1 minute), and extension (72%C; 90 seconds), except during the first cycle in which the denaturing time is increased to 5 minutes, and during the last cycle in which the extension time is increased to 5 minutes.

PCR products are purified using Qia-quick® PCR purification kits (Qiagen, cat# 28104; Chatsworth, Calif.). Yield and purity of the PCR product determined spectrophotometrically at OD₂₆₀ on a Beckman DU 650 spectrophotometer.

2. Dideoxy Sequence Analysis

Fluorescent dye is attached to PCR products for automated sequencing using the Taq Dye Terminator® Kit (Perkin-Elmer cat# 401628). DNA sequencing is performed in both forward and reverse directions on an Applied Biosystems, Inc. (ABI) Foster City, Calif.., automated Model 377® sequencer. The software used for analysis of the resulting data is “sequence Navigator® software” purchased through ABI. The BRCA1^{(omi2)} SEQ. ID. NO.:3 sequence is entered into the Sequence Navigator® software as the Standard for comparison. The Sequence Navigator® software compares the sample sequence to the BRCA1^{(omi2)} SEQ. ID. NO.:3 standard, base by base. The Sequence Navigator® software highlights all differences between the BRCA1^{(omi2)} SEQ. ID. NO.:3 DNA sequence and the patient's sample sequence.

A first technologist checks the computerized results by comparing visually the BRCA1^{(omi2)} SEQ. ID. NO.:3 standard against the patient's sample, and again highlights any differences between the standard and the sample. The first primary technologist then interprets the sequence variations at each position along the sequence. Chromatograms from each sequence variation are generated by the Sequence Navigator® software and printed on a color printer. The peaks are interpreted by the first primary technologist and also by a second primary technologist. A secondary technologist then reviews the chromatograms. The results are finally interpreted by a geneticist. In each instance, a variation is compared to known polymorphisms for position and base change. If the sample BRCA1 sequence matches the BRCA1^{(omi2)} SEQ. ID. NO.:3 standard, with only variations within the known list of polymorphisms, it is interpreted as a gene sequence.

### Example 4

### Determining the Presence of a Mutation in the BRCA1 Gene using BRCA1^{(omi)} and Seven Polymorphisms for Reference

A person skilled in the art of genetic susceptibility testing will find the present invention useful for determining the presence of a known or previously unknown mutation in the BRCA1 gene. A list of mutations of BRCA1 is publicly available in the Breast Cancer Information Core at:

http://www.nchgr.nih.gov/dir/lab_transfer/bic. This data site became publicly available on Nov. 1, 1995. Friend, S. et al. Nature Genetics 11:238, (1995). In this example, a mutation in exon 11 is characterized by amplifying the region of the mutation with a primer which matches the region of the mutation.

Exon 11 of the BRCA1 gene is subjected to direct dideoxy sequence analysis by asymmetric amplification using the polymerase chain reaction (PCR) to generate a single stranded product amplified from this DNA sample. Shuldiner, et al, Handbook of Techniques in Endocrine Research, p. 457-486, DePablo, F., Scanes, C., eds., Academic Press, Inc., 1993. Fluorescent dye is attached for automated sequencing using the Taq Dye Terminator® Kit (Perkin-Elmer cat# 401628). DNA sequencing is performed in both forward and reverse directions on an Applied Biosystems, Inc. (ABI) automated Model 377(® sequencer. The software used for analysis of the resulting data is “Sequence Navigator® software” purchased through ABI.

1. Polymerase Chain Reaction (PCR) Amplification

Genomic DNA (100 nanograms) extracted from white blood cells of the subject is amplified in a final volume of 25 microliters containing 1 microliter (100 nanograms) genomic DNA, 2.5 microliters 10×PCR buffer (100 mM Tris, pH 8.3, 500 mM KCl, 1.2 mM MgCl₂), 2.5 microliters 10×dNTP mix (2 mM each nucleotide), 2.5 microliters forward primer (BRCA1-11K-F, 10 micromolar solution), 2.5 microliters reverse primer (BRCA1-11K-R, 10 micromolar solution),and 1 microliter Taq polymerase (5 units), and 13 microliters of water.

The PCR primers used to amplify segment K of exon 11 (where the mutation is found) are as follows:

| | |
|---|---|
| BRCA1-11K-F: | |
| 5′-GCA AAA GCG TCC AGA AAG GA-3′ | SEQ ID NO:69 |
| BRCA1-11K-R: | |
| 5′-AGT CTT CCA ATT CAC TGC AC-3′ | SEQ ID NO:70 |

The primers are synthesized on an DNA/RNA Model 394® Synthesizer. Thirty-five cycles are performed, each consisting of denaturing (95% C; 30 seconds), annealing (55% C; 1 minute), and extension (72% C; 90 seconds), except during the first cycle in which the denaturing time is increased to 5 minutes, and during the last cycle in which the extension time is increased to 5 minutes.

PCR products are purified using Qia-quick® PCR purification kits (Qiagen, cat# 28104; Chatsworth, Calif.). Yield and purity of the PCR product determined spectrophotometrically at OD₂₆₀ on a Beckman DU 650 spectrophotometer.

2. Dideoxy Sequence Analysis

Fluorescent dye is attached to PCR products for automated sequencing using the Taq Dye Terminator® Kit (Perkin-Elmer cat# 401628). DNA sequencing is performed in both forward and reverse directions on an Applied Biosystems, Inc. (ABI) Foster City, Calif.., automated Model 377® sequencer. The software used for analysis of the resulting data is “Sequence Navigator® software” purchased through ABI. The BRCA1^{(omi2)} SEQ. ID. NO.:3 sequence is entered into the Sequence Navigator® software as the Standard for comparison. The Sequence Navigator® software compares the sample sequence to the BRCA1^{(omi2)} SEQ. ID. NO.:3 standard, base by base. The Sequence Navigator® software highlights all differences between the BRCA1^{(omi2)} SEQ. ID. NO.:3 DNA sequence and the patient's sample sequence.

A first technologist checks the computerized results by comparing visually the BRCA1^{(omi2)} SEQ. ID. NO.:3 standard against the patient's sample, and again highlights any differences between the standard and the sample. The first primary technologist then interprets the sequence variations at each position along the sequence. Chromatograms from each sequence variation are generated by the Sequence Navigator® software and printed on a color printer. The peaks are interpreted by the first primary technologist and a second primary technologist. A secondary technologist then reviews the chromatograms. The results are finally interpreted by a geneticist. In each instance, a variation is compared to known polymorphisms for position and base change. Mutations are noted by the length of non-matching variation. Such a lengthy mismatch pattern occurs with deletions and substitutions.

3. Result

Using the above PCR amplification and standard fluorescent sequencing technology, The 3888delGA mutation may be found. The 3888delGA mutation The BRCA1 gene lies in segment “K” of exon 11. The DNA sequence results demonstrate the presence of a two base pair deletion at nucleotides 3888 and 3889 of the published BRCA1^{(omi)} sequence. This mutation interrupts the reading frame of the BRCA1 transcript, resulting in the appearance of an in-frame terminator (TAG) at codon position 1265. This mutation is, therefore, predicted to result in a truncated, and most likely, non-functional protein. The formal name of the mutation will be 3888delGA. This mutation is named in accordance with the suggested nomenclature for naming mutations, Baudet, A et al., Human Mutation 2:245-248, (1993).

### Example 5

### Generation of Monoclonal and Polyclonal Antibodies using GST-BRCA1^{(omi)} Fusion Protein as an Immunogen

DNA primers are used to amplify a fragment of BRCA1^{(omi1)} cDNA (SEQ. ID. NO:1, 3, or 5) using PCR technology. The product is then digested with suitable restriction enzymes and fused in frame with the gene encoding glutathione S-transferase (GST) in Escherichia coli using GST expression vector pGEX (Pharmacia Biotech Inc.) The expression of the fusion protein is induced by the addition of isopropyl-β-thiogalactopyranoside. The bacteria are then lysed and the overexpressed fusion protein is purified with glutathione-sepharose beads. The fusion protein is then verified by SDS/PAGE gel and N-terminus protein sequencing. The purified protein is used to immunize rabbits according to standard procedures described in Harlow & Lane (1988). Polycolonal antibody is collected from the serum several weeks after and purified using known methods in the art. Monoclonal antibodies against all or fragments of BRCA1^{(omi)} protein, polypeptides, or functional equivalents are obtained using hybridoma technology, see Harlow & Lane (1988). The BRCA1^{(omi)} protein or polypeptide is coupled to the carrier keyhole limpet hemocyanin in the presence of glutaraldehyde. The conjugated immunogen is mixed with an adjuvant and injected into rabbits. Spleens from antibody-containing rabbits are removed. The B-cells isolated from spleen are fused to myeloma cells using polyethylene glycol (PEG) to promote fusion. The hybrids between the myeloma and B-cells are selected and screened for the production of antibodies to immunogen BRCA1^{(omi)} protein or polypeptide. Positive cells are recloned to generate monoclonal antibodies.

### Example 6

### Detection of BRCA1^{(omi)} Expression in Human Tissues and Cell Lines

The expression of BRCA1^{(omi)} in human tissues is determined using Northern blot analysis. Human tissues include those from pancreas, testis, prostate, ovary, breast, small intestine, and colon are obtained from Clontech Laboratories, Inc., Palo Alto, Calif. The poly(A)⁺ mRNA Northern blots from different human tissues is hybridized to BRCA1^{(omi)} cDNA probes according to the manufacture protocol. The expression level is further confirmed by RT-PCR using oligo-d(T) as a primer and other suitable primers.

For Northern Blot analysis of cancer cell lines, the human ovarian cancer cell line SKOV-3 and the human breast cancer cell line MCF-7 are obtained from the American Type Culture Collection. Total RNA is prepared by lysing cell in the presence of guanidinium isocyanate. Poly(A)⁺ mRNA is isolated using the PolyATract mRNA isolation system from Promega, Madison, Wis. The isolated RNA is then electrophoresed under denaturing conditions and transferred to Nylon membrane. The probe used for Northern blot is a fragment of BRCA1^{(omi)} sequence obtained by PCR amplification. The probes are labeled with [α-³²P] dCTP using a random-primed labeling kit (Amersham Life Science, Arlington Heights, Ill.).

### Example 7

### Expression of BRCA1^{(omi)} Protein

The whole-cell extracts of BRCA1^{(omi)} transfected cells are subjected to immunoprecipitation and immunoblotting to determine the BRCA1^{(omi)} protein level. The BRCA ₁^{(omi)} protein or polypeptide is immmunoprecipitated using anti-BRCA1 antibodies prepared according to Example 4 or purchased from Santa Cruz Biotechnology Inc. Samples are then fractionated using SDS/PAGE gel and transferred to nitrocellulose. Western immunoblotting of the BRCA1^{(omi)} protein is performed with the indicated antibodies. Antibody reaction is revealed using enhanced chemiluminescence reagents (Dupont New England Nuclear, Boston, Mass.).

### Example 8

### Use of the BRCA1^{(omi1)} Gene Therapy

The growth of ovarian or breast cancer may be arrested by increasing the expression of the BRCA1 gene where inadequate expression of that gene is responsible for hereditary ovarian or breast cancer. It has been demonstrated that transfection of BRCA1 into cancer cells inhibits their growth and reduces tumorigenesis. Gene therapy is performed on a patient to reduce the size of a tumor. The LXSN vector is transformed with any of the BRCA1^{(omi1)} SEQ. ID. NO.:1, BRCA1^{(omi2)} SEQ. ID. NO.:3, or BRCA1^{(omi3)} SEQ. ID. NO.:5 coding region.

### Vector

The LXSN vector is transformed with wildtype BRCA1^{(omi1)} SEQ. ID. NO.:1 coding sequence. The LXSN-BRCA1^{(omi1)} retroviral expression vector is constructed by cloning a SalI-linkered BRCA1^{(omi1)} cDNA (nucleotides 1-5711) into the XhoI site of the vector LXSN. Constructs are confirmed by DNA sequencing. Holt et al. Nature Genetics 12: 298-302 (1996).

Retroviral vectors are manufactured from viral producer cells using serum free and phenol-red free conditions and tested for sterility, absence of specific pathogens, and absence of replication-competent retrovirus by standard assays. Retrovirus is stored frozen in aliquots which have been tested.

Patients receive a complete physical exam, blood, and urine tests to determine overall health. They may also have a chest X-ray, electrocardiogram, and appropriate radiologic procedures to assess tumor stage.

Patients with metastatic ovarian cancer are treated with retroviral gene therapy by infusion of recombinant LXSN-BRCA1^{(omi)} retroviral vectors into peritoneal sites containing tumor, between 10⁹ and 10¹⁰ viral particles per dose. Blood samples are drawn each day and tested for the presence of retroviral vector by sensitive polymerase chain reaction (PCR)-based assays. The fluid which is removed is analyzed to determine:

1. The percentage of cancer cells which are taking up the recombinant LXSN-BRCA1^{(omi1)} retroviral vector combination. Successful transfer of BRCA1 gene into cancer cells is shown by both RT-PCR analysis and in situ hybridization. RT-PCR is performed with by the method of Thompson et al. Nature Genetics 9: 444-450 (1995), using primers derived from BRCA1^{(omi1)} SEQ. ID. NO.:1. Cell lysates are prepared and immunoblotting is performed by the method of Jensen et al. Nature Genetics 12: 303-308 1996) and Jensen et al. Biochemistry 31: 10887-10892 (1992).

2. Presence of programmed cell death using ApoTAG® in situ apoptosis detection kit (Oncor, Inc., Gaithersburg, Md.) and DNA analysis.

3. Measurement of BRCA I gene expression by slide immunofluorescence or western blot.

Patients with measurable disease are also evaluated for a clinical response to LXSN-BRCAI, especially those that do not undergo a palliative intervention immediately after retroviral vector therapy. Fluid cytology, abdominal girth, CT scans of the abdomen, and local symptoms are followed.

For other sites of disease, conventional response criteria are used as follows:

1 Complete Response (CR), complete disappearance of all measurable lesions and of all signs and symptoms of disease for at least 4 weeks.

2. Partial Response (PR), decrease of at least 50% of the sum of the products of the 2 largest perpendicular diameters of all measurable lesions as determined by 2 observations not less than 4 weeks apart. To be considered a PR, no new lesions should have appeared during this period and none should have increased in size.

3. Stable Disease, less than 25% change in tumor volume from previous evaluations.

4. Progressive Disease, greater than 25% increase in tumor measurements from prior evaluations.

The number of doses depends upon the response to treatment.

For further information related to this gene therapy approach see in “BRCA1 Retroviral Gene Therapy for Ovarian Cancer” a Human Gene Transfer Protocol: NIH ORDA Registration #: 9603-149 Jeffrey Holt, J T, M.D. and Carlos L. Arteaga, M.D.

### Example 9

### Protein Replacement Therapy

Therapeutically elevated level of functional BRCA1 protein may alleviate the absence or reduced endogenous BRCA1 tumor suppressing activity. Breast or ovarian cancer is treated by the administration of a therapeutically effective amount of BRCA1^{(omi)} protein, a polypeptide, or its functional equivalent in a pharmaceutically acceptable carrier. Clinically effective delivery method is applied either locally at the site of the tumor or systemically to reach other metastasized locations with known protocols in the art. These protocols may employ the methods of direct injection into a tumor or diffusion using time release capsule. A therapeutically effective dosage is determined by one of skill in the art. Breast and ovarian cancer may be prevented by the administration of a prophylactically effective amount of the BRCA1^{(omi)} protein, polypeptide, or its functional equivalent in a pharmaceutically acceptable carrier. Individuals with known risk for breast or ovarian cancer are subjected to protein replacement therapy to prevent tumorigenesis or to decrease the risk of cancer. Elevated risk for breast and ovarian cancer includes factors such as carriers of one or more known BRCA1 and BRCA2 mutations, late child bearing, early onset of menstrual period, late occurrence of menopause, and certain high risk dietary habits. Clinically effective delivery method is used with known protocols in the art, such as administration into peritoneal cavity, or using an implantable time release capsule. A prophylactically effective dosage is determined by one of skill in the art.

### Table of References

1. Sanger, F., et al, J. Mol. Biol. 42:1617, (1980).

2. Beaucage, et al., Tetrahedron Letters 22:1859-1862, (1981).

3. Maniatis, et. al. in Molecular Cloning:A Laboratory Manual, Cold Spring Harbor, N.Y., p 280-281, (1982).

4. Conner, et. al., Proc. Natl. Acad. Sci. U.S.A. 80:278, (1983)

5. Saiki, et. al., Bio/Technology 3:1008-1012, (1985)

6. Landgren, et. al., Science 241:1007,(1988)

7. Landgren, et. al., Science 242 :229-237, (1988).

8. PCR. A Practical Approach, ILR Press, Eds. M. J. McPherson, P. Quirke, and G. R. Taylor, (1992).

9. Easton et al., American Journal of Human Genetics 52:678-701, (1993).

10. U.S. Pat. No. 4,458,066.

11. Rowell, S., et al., American Journal of Human Genetics 55:861-865, (1994)

12. Miki, Y. et al., Science 266:66-71, (1994).

13. Friedman, L. et al., Nature Genetics 8:399-404, (1994).

14. Baudet, A et al., Human Mutation 2:245-248, (1993).

15. Friend, S. et al., Nature Genetics 11:238, (1995).

16. Arteaga, C L and J T Holt Cancer Research 56:1098-1103 (1996).

17. Holt, J T et al., Nature Genetics 12:298-302 (1996).

18. Jensen, R A et al., Nature Genetics 12:303-308 (1996).

19. Steeg, P. Nature Genetics 12:223-225 (1996).

20. Thompson, M E et al., Nature Genetics 9: 444-450 (1995)

21. Holt, Jn T, and C. Arteaga, Gene Therapy Protocol ORDA #: 9602-148 and 9603-149 ORDA approved Protocol for BRCA1 Gene Therapy.

22. Sambrook, et al., Molecular Cloning: A Laboratory Manual, Second Edition, Cold Spring Harbor Laboratory, Cold Spring Harbor, N.Y. (1989).

23. Bertwistle and Ashworth, Curr. Opin. Genet. Dev. 8(1): 14-20 (1998).

24. Zhang et al., Cell 92:433-436 (1998).

25. Sharan et al., Nature 386:804-810 (1997).

26. Katagiri et al., Genes, Chromosomes & Cancer 21:217-222 (1988).

27. Crooke, Annu. Rev. Pharmacol. Toxicol. 32:329-376 (1992)

28. Robinson-Benion and Holt, Methods Enzymol. 254:363-375 (1995).

29. Harlow & Lane, Antibodies: A Laboratory Manual, Cold Spring Harbor Laboratory, Cold Spring Harbor, N.Y., 1988.

30. Shuldiner, et al., Handbook of Techniques in Endocrine Research, p. 457-486, DePablo, F., Scanes, C., Eds., Academic Press, Inc., 1993.

31. Holt et al., Nature Genetics 12: 298-302 (1996).

32. Thompson et al., Nature Genetics 9: 444-450 (1995).

33. Jensen et al., Nature Genetics 12: 303-308 (1996)

34. Jensen et al., Biochemistry 31: 10887-10892 (1992).

35. U.S. Pat. No. 5,593,840

36. U.S. Pat. No. 5,445,934

37. U.S. Pat. No. 5,510,270

38. U.S. Pat. No. 5,547,839

39. Kenneth W. Culver, M. D in Gene Therapy A Primer For Physicians 2d Ed. Mary Ann Liebert Inc. (1996).

40. Husain et al., Cancer Res. 58:1120-1123 (1998).

41. Ruffler et al., Proc. Natl. Acad. Sci. USA 94:7138-7143 (1997)

Although the invention has been described with reference to the presently preferred embodiments, it should be understood that various modifications can be made without departing from the spirit of the invention. Accordingly, the invention is limited only by the following claims.

We claim:

1. A protein sequence comprising an amino acid sequence derived from the BRCA1^{(omi1)} sequence as set forth in SEQ. ID. NO:2.

2. A protein sequence comprising an amino acid sequence derived from the BRCA1^{(omi2)} sequence as set forth in SEQ. ID. NO:4.

3. A protein sequence comprising an amino acid sequence derived from the BRCA1^{(omi3)} sequence as set forth in SEQ. ID. NO:6.

4. A method of determining the consensus genomic sequence or consensus coding sequence for a target gene, comprising:
a) screening a number of individuals in a population for a family history which indicates inheritance of normal alleles for a target gene;
b) isolating at least one allele of the target gene from individuals found to have a family history which indicates inheritance of normal alleles for a target gene; c) sequencing each allele;
d) comparing the nucleic acid sequence of the genomic sequence or of the coding sequence of each allele of the target gene to determine similarities and differences in the nucleic acid sequence; and
e) determining which allele of the target gene occurs with the greatest frequency.

5. A plurality of oligonucleotide probes each capable of hybridizing to a sample BRCA1^{(omi)} gene having the sequence as set forth in SEQ. ID. NO:1, 3 and 5, or a sequence complementary thereto.

6. A chip array having “n” elements for performing allele specific sequence-based techniques comprising a solid phase chip and oligonucleotides having “n” different nucleotide sequences,
wherein “n” is an interger greater than or equal to seven,
wherein said oligonucleotides are bound to said solid phase chip in a manner which permits said oligonucleotides to effectively hybridize to complementary oligonucleotides or polynucleotides,
wherein oligonucleotides having different nucleotide sequence are bound to said solid phase chip at different locations so that a particular location on said solid phase chip exclusively binds oligonucleotides having a specific nucleotide sequence, and
wherein oligonucleotides are capable of specifically hybridizing to the BRCA1^{(omi)} DNA having the sequence as set forth in SEQ. ID. NO:1, 3 and 5, or a sequence complementary thereto.

7. A method of performing gene therapy on a patient, comprising:
a) contacting cancer cells in vivo with an effective amount of a vector comprising DNA containing at least a portion of BRCA1^{(omi)} coding sequence as set forth in SEQ. ID. NO:1, 3 or 5, or a sequence complementary thereto;
b) allowing the vector to enter the cancer cells; and
c) measuring a reduction in tumor growth.

8. The method according to claim 7 wherein said cancer cells have a mutation in a BRCA1 gene.

9. The method according to claim 7 wherein said patient has a mutation in the BRCA1 gene of non-cancer cells.

10. A method of performing gene therapy on a patient or a sample, comprising:
a) contacting cells in vivo or in vitro with an effective amount of a vector comprising DNA containing at least a portion of BRCA1^{(omi)} coding sequence as set forth in SEQ ID NO:1, 3 or 5 or a sequence complementary thereto; and
b) allowing the vector to enter the cells,
wherein said patient has a reduced susceptibility for developing a cancer associated with a mutation in the BRCA1 gene.

11. A method according to claim 10 wherein said cells include healthy breast, ovarian, prostate, and colon tissues.

12. A method according to claim 10 wherein a patient has an inherited mutation in the BRCA1 gene.

13. A method of treating a patient suspected of having a tumor, comprising:
a) administering an effective amount of BRCA1 tumor growth inhibitor having an amino acid sequence derived from SEQ ID NO:2, 4, or 6, to a patient;
b) allowing the patient's cells to take up the protein, and
c) measuring a reduction in tumor growth.

14. The method according to claim 13 wherein said tumor is a breast cancer, an ovarian cancer, a prostate, or a colon cancer.

15. The method according to claim 13 wherein said patient has an inherited mutation in a BRCA1 gene.

16. A method of preventing the formation or growth of a tumor, comprising:
a) administering an effective amount of BRCA1 tumor growth inhibitor having an amino acid sequence derived from SEQ ID NO:2, 4, or 6, to a patient suspected of having an inherited mutation in a BRCA1 gene; and
b) allowing the patient cells to take up the protein.

17. The method according to claim 16 wherein the protein is administered parenternally, by buccal adsorption or inhalation.

18. A cloning vector comprising:
(a) a DNA sequence as set forth in SEQ. ID. NO:1, SEQ. ID. NO:3, SEQ. ID. NO:5, or any fragments thereof; and
(b) one or more suitable regulatory sequences to induce replication and/or integration in a host cell.

19. An expression vector comprising a DNA sequence as set forth in SEQ. ID. NO:1, SEQ. ID. NO:3, SEQ. ID. NO:5, or any fragments thereof operatively linked to one or more promoter sequences capable of directing expression of said sequence in a host cell.

20. A host cell transformed with the vector according to any one of the claims 18 and 19.

21. A BRCA1 polypeptide which is selected from the group consisting of:
(a) a fragment of BRCA1^{(omi)} protein sequence as set forth in SEQ. ID. NO:2, SEQ. ID. NO:4, or SEQ. ID. NO:6;
(b) an amino acid sequence which is substantially homologous to the BRCA1^{(omi)} protein sequence as set forth in SEQ. ID. NO:2, SEQ. ID. NO:4, or SEQ. ID. NO:6;
(c) a molecule which has similar function to the BRCA1^{(omi)} protein; and
(d) a fusion protein of (a), (b), or (c).

22. An anti-BRCA1 antibody wherein a molecule according to any one of the claims 1-3, and 21 is used as an immunogen.

23. A diagnostic reagent comprising a molecule selected from the group consisting of:
(a) a DNA sequence as set forth in SEQ. ID. NO:1, SEQ. ID. NO:3, SEQ. ID. NO:5, or a sequence complementary thereto;
(b) a nucleic acid fragment of (a) comprising at least 10 nucleotide in length;
(c) a sequence which hybridizes to (a) or (b);
(d) a polypeptide according to any on eof the claims 1-3 and 21; and
(e) an antibody which specifically binds to the polypeptide of (d).

24. A pharmaceutical composition comprising a molecule according to any one of the claims 1-3 and 21 in a pharmaceutically acceptable carrier.

25. A pharmaceutical composition comprising a molecule according claim 22 in a pharmaceutically acceptable carrier.

26. A pharmaceutical composition comprising a molecule according to claim 23 in a pharmaceutically acceptable carrier.
